(19) United States
(12) Patent Application Publication
(10) Pub. No.: US 2008/0139510 A1
(43) Pub. Date: Jun. 12, 2008

(54) Treatment of migraine headaches with sublingual amino acids

(76) Inventor: Abe Rose, Houston, TX (US)

Correspondence Address: ABE ROSE, 10300 HARWIN DRIVE #1222, HOUSTON, TX, 77038, US

(21) Appl. No.: 11/608,180

(22) Filed: Dec. 7, 2006

(51) Int. Cl. A61K 31/675; A61P 25/00; A61K 31/405

(52) U.S. Cl. 514/89; 514/419

(57) ABSTRACT

The invention relates to compositions and methods for treating, or managing the acute pain of a classic migraine headache through the combined-sublingual administration-of therapeutically effective amounts of amino acids, and vitamin cofactors. By bypassing gut metabolism common through conventional oral administration, bio-active nutrients are transfered directly to the central nervous system, synergistically restoring the deficient inhibitory tone that precipitates a classic migraine attack, alleviating headache symptoms.

### BACKGROUND OF THE INVENTION

1. Field of the Invention:

In general, the present invention relates to the field of amino acid therapy. Specifically, the invention relates to a technology supporting and enhancing the serotonin and GABA neurotransmitter systems in humans. Most specifically, the invention relates to safe, effective compositions, methods, and therapies for treating and ameliorating the pain associated with migraine.

2. Prior Art

The Etiology of migraines is not yet fully understood. There is however, found in the large body of scientific research, certain biochemical mechanisms repeatedly implicated in the Pathophysiology of both migraine with aura and migraine without. Migraine with aura, herein to be known as classic migraine, is distinguishable from migraine with out aura, herein to be known as common migraine, by it's onset symptoms.

The pain phase of a classic migraine is preceded by an aura, a visual disturbance that occupies the patients field of vision. This disturbance usually consists of a dual aspect, comprising a blind spot (scotoma) located inside the central field of vision, and an adjacent flashing light in the form of a zigzag line (fortification spectrum) at the periphery. Initially appearing barely noticeable, may gradually broaden to encompass the patients entire field of vision producing a transient and partial blindness, and can persist from between five minutes to under an hour before subsiding, and vision is restored again. After a period of about five to thirty minutes, where the patient seems to experience a remission of symptoms, a phase of intense cranial pain accompanied by nausea and sensitivity to both light and sound occurs, which can then last for any number of hours.

Sufferers of classic migraines in contrast to sufferers experiencing common migraines and control subjects, have consistently show lower serotonin levels between episodes, suggesting a hypofunction of the serotonin system (Nagata et al., 2006). This hypofunction may indicate an inadequacy of the enzymatic synthesis of serotonin, down regulation of the 5-HT receptor, or increased catecholaminergic activity, by any number of factors, be they hereditary, behavioral, or dietary. After onset of migraine however, Sufferers of classic migraines in contrast to sufferers experiencing common migraines and control subjects, have repeatedly presented higher plasma serotonin levels during attacks (Ferrari et al., 1989).

It is postulated, that classic migraine pain, is a consequence of biochemical conditions that result from the aura itself. The au effects the CNS by stimulating the trigeminal nociceptive system through a cortical excitability/depression wave, potentiated initially by depleted CNS serotonin (Supornsilpchai et al., 2006). Stimulated trigeminovascular fibers then release vasoactive neuropeptides that initiate neurogenic inflammation (Ducros A., 2006). There is a further elevation in the ratio of dopaminergic to noradrenergic activity caused by a concomitant sympathetic hypo-dysfunction (Peroutka S J., 2004). The additional release of 5-HT from aggregated platelets in response to inflammatory stress (Jha et al., 1992), and a lack of 5-HT degradation by a decrease of MAO enzyme (Caramona et al., 1990), manifests a transient elevation of plasma serotonin with a collateral low CNS serotonin.

Serotonin levels are also increased in the gut during migraine aura, subsequent to enhanced enteric serotonin synthesis and release. This is apparent by the occurrence of nausea that typically accompanies classic migraines. After onset of migraine, mucosal enterochromaffin cells release 5-HT; this stimulates 5-HT3 receptors on adjacent vagal afferent nerves. The depolarization of the vagal afferent nerves then stimulates the vomiting center in the brainstem inducing emesis, or vomiting urgency (Endo et al., 2000). Epidemiological studies reveal that over 90% of patients experienced nausea during a migraine attack (Gladstone, et al., 2003).

It is of clinical significance, the method of ameliorating the pain of a migraine by restoring CNS levels of serotonin. The success the pharmaceutical triptans, or serotonin agonists, support the hypothesis that if serotonergic tone is restored to the CNS (5-HT1 DR), headache pain can be alleviated. As such, triptans are currently the most successful, popular, and prescribed of all the migraine pharmaceuticals, and represent a significant focus of the prior art (U.S. Pat. No. 5,872,145; U.S. Pat. No. 5,891,885; U.S. Pat. No. 6,060,499; U.S. Pat. No. 6,255,334; U.S. Pat. No. 6,380,226; U.S. Pat. No. 6,380,242; U.S. Pat. No. 6,476,042, etc.).

Many triptans are available in oral preparations: sumatriptan, rizatriptan, zolmitriptan, naratriptan, almotriptan, frovatriptan eletriptan (Gladstone et al., 2003). Triptans often vary from each other in their efficacy (Ferrari et al., 2001), and pose the occasional risk of drug-drug interactions (Armstrong et al., 2002). They are also generally contraindicated in patients with cardiac or cerebrovascular disease for their actions as vasoconstrictors (Jamieson D G., 2002). Although touted for their generally high safety, one study concluded that more than half of migraine patients do not use triptans again after their first experience with the drug (Ifergane et al., 2006). An investigation of the prior art also reveals “It is known that triptans do not work well in 20-25% of the patients and fail 40% of the time in patients who have earlier responded well to this class of drugs” (U.S. Pat. No. 7,070,817).

Characteristically, an agonist or analog will function as a neurotransmitter by similarity, binding to it's receptor, activating signal transduction. The initial need for these agonists in the first place, is due contingently to the depleted synaptic levels of neurotransmitter which they simulate, as is the case with triptans in serotonin neurochemistry.

It is stated however, that recurrence of headache within 24 hours after an initial successful response with triptans occurs in a significant number of treated patients (Tfelt-Hansen et al., 2000). Subsequent rebound headaches can also occur from the overuse of triptans for various reasons: central sensitisation from repetitive activation of nociceptive pathways; a direct effect of the medication on the capacity of the brain to inhibit pain; a decrease in blood serotonin due to repetitive medication administration with attendant upregulation of serotonin receptors; cellular adaptation in the brain, etc. (Smith et al., 2004). Treatment with a triptan usually only temporarily distorts the basic pattern of the attacks (Linde et al., 2006). Recurrence of headache with triptans is conceivable because serotonin levels are never really restored to the nervous system or cerebrospinal fluid. Symptoms are alleviated because of receptor binding of a serotonin analog, rendering only a transient optimization of serotonin tone. Once molecularly deactivated or metabolized, triptans could reveal once again the same fundamental precondition of the migraine-low CNS serotonin.

In an attempt to avoid the adverse effects or expense of pharmaceutical therapies, many individuals have sought out more natural or alternative treatment regimes, including or combining: magnesium, ginger, ginko biloba, feverfew, and melatonin, to name a few (U.S. Pat. No. 6,068,999). Many of these preparations may have anti-inflammatory or analgesic action, or may directly effect the indolamine pathway. The results have not been consistent, or always significant ((Maizels et al., 2004), (Pfaffenrath et al., 1996)). Some have even advocated the use of tobacco (U.S. Pat. No. 7,070,817). In keeping, However, with the same effective philosophy behind the triptans: a treatment that addresses the aggressive restoration of serotonergic tone -after onset of migraine headache-yields the most reasonable and reliable solution.

Oral administration of the amino acid 5-HTP, in tablet or capsule form, is the commonly accepted method of administration of 5-HTP, and remains an available and effective way to elevate serotonin (5-HT) (Birdsall T C., 1998). It is also known in the prior art, the prophylactic use of 5-HTP in treating stress (U.S. Pat. No. 6,579,899). In the treatment of migraine, however, Clinical evidence supports it's only moderate effectiveness, but remarkable safety (De Benedittis et al., 1985). Results indicate the primary benefits of 5-HTP lie in it's routine daily use as a preventative (U.S. Pat. No. 5,939,076), demonstrating it's ability to minimize migraine frequency ((Maissen et al., 1991), (Ribeiro C A., 2000)), and in some cases, headache intensity and duration (Titus et al., 1986). Repeatedly however, 5-HTP has been shown to be ineffective in aborting or significantly mitigating pain after onset of migraine attack. The problem lies in the peripheral metabolism of 5-HTP by the enteric nervous system (ENS), common with oral delivery, permitting less bioavailability of serotonin precursors to the CNS (Turner et al., 2006)—where it is needed most.

### STATEMENT OF THE PROBLEM

Commercial 5-HTP is widely available as an oral supplement. Since 5-HTP is well absorbed from an oral dose, further benefits of a sublingual supplement might seem commercially and remedially unnecessary. As such, there is a paucity of sublingual 5-htp preparations commercially available.

Generally, oral 5-HTP increase both systemic 5-HTP and serotonin; It is not usually known though, by the amount of an ingested dose, how much 5-HTP will be metabolized to serotonin in the gut, liver, or elsewhere in the periphery, and how much 5-HTP makes it to the brain via systemic blood circulation. Through daily use, as both the peripheral and central nervous system become exposed to the amino acid, an alteration of the basal catecholamine serotonin ratio is gradually achieved. Accordingly, clinical trials have often demonstrated 5-HTP, a suitable daily prophylactic, to be ineffective as an abortive migraine treatment -even at large oral doses.

Migraine induces beta-adrenergic activation, causing serotonin release from intestinal enterochromaffin cells, decreasing there intracellular content; additionally ingested 5-HTP is taken back up into enterochromaffin cells where further decarboxylation to serotonin takes place locally. The success of a 5-HTP preparation in aborting migraine pain is contingent upon a delivery that can facilitate it's timely conversion to serotonin centrally. No where in the archives of current published medical research, nor is it an object of the prior art or market, the proposed method and composition of the amino acid 5-Hydroxytryptophan and necessary coenzymes for the abortive treatment of classic migraine by an alternative sublingual administration.

### SUMMARY OF THE INVENTION

It is an object of this invention, the preparation and administration of a novel medicament for the treatment of acute migraine pain. The treatment restores depleted serotonin levels precipitating the attack, through a delivery system that not only provides optimal transport of serotonin precursors to the central nervous system, but supply the necessary synergists that support and enhance the entire indolamine pathway. A subsequent benefit of this treatment is a decline in the frequency of attacks secondary to replenished basal serotonin levels.

In a broad aspect within the scope of the present invention, the embodiment shall include the combined ingredients of: The amino acid 5-Hydroxy L-Tryptophan in an effective therapeutic amount of approximately 100 mg. Vitamin B6 in an effective therapeutic amount of approximately 20 mg. Vitamin C in an effective therapeutic amount of approximately 500 mg.

In a more specific aspect within the scope of the present invention, the embodiment shall include, but not be limited to, the add ingredients of: The amino acid Gamma-Aminobutyric Acid in an effective therapeutic amount of approximately 100 mg. Vitamin B3 in an effective therapeutic amount of approximately 100 mg.

The above constituents represent the active ingredients, which are to be combined and mixed in powdered form. The elective inclusion of inactive ingredients are discussed later.

It is a method of this invention, that the preparation should be administered to a human, after complete cessation of the migrain aura and the initial onset of the pain phase has begun. The preparation of the invention shall be administered sublingually, preferably in the form of either a loose or coherent powdered medicament, to facilitate quicker dissolution of the preparation, and retained until the preparation has dissolved. Administration of the medicament produces a neural inhibition of trigeminal nerve excitation, followed by a progressive reduction in cranial sensitivity without added need of analgesics, anti-inflammatory's, or serotonin agonists. With the stated composition, and timing of administration, the applicant has facilitated the successful and complete remission of the cranial pain and associated symptoms of classic migraine within one to two hours of onset, With outstanding safety, consistency, and repeatability.

### DISCUSSION OF MATERIALS AND COMPOSITIONS

### 5-HTP

The neurotransmitter serotonin can not be administered systemically to elevate CNS neurotransmitters, as it cannot pass the blood-brain-barrier. As a preferred alternative, the use of the amino acid 5-hydroxytryptophan (5-HTP) is warranted. 5-HTP is the immediate precursor of serotonin (5-HT), and readily passes the blood-brain-barrier.

The conventional oral administration of the amino acid 5-HTP to increase central and peripheral serotonin levels is well appreciated. However, it suffers in it's delivery method to effect a consistent satisfactory clinical resolution to the severe symptoms of a classic migrain-during an attack. Furthermore, additional 5-HTP after migraine onset-through oral administration-further increases the intestinal serotonin precursor pool during a time of already enhanced serotonin synthesis and release by the ENS, further exacerbating nausea, while preventing the complete or effectual availability of serotonin precursors to the CNS.

It is an object of this invention, that the sublingual administration of the amino acid 5-HTP prevents it's intestinal metabolism, and thus, further activation of serotonin at the enteric neurons and enterochromaffin cells of the mucosa, where 90% of the body's serotonin is stored. Moreover, there is the additional bypass of the liver's “first pass” effect, where significantly more decarboxylation of 5-HTP would normally take place. 5-HTP administered beneath the tongue is diffused into the blood vessels of the sublingual and buccal mucosa, where it is osmotically taken up into the circulatory system. The addition of a buffering system within the preparation, to lower oral pH and enhance absorption, shall be incorporated. This will render a prompt systemic supply of the necessary serotonin precursor and enzyme cofactors directly to the brain via the sublingual, lingual, and carotid arteries. There, 5-HTP easily passes the blood brain barrier with the water soluble vitamin cofactors, where the CNS synthesis of serotonin is facilitated.

5-HTP is extracted from the Griffonia Simplicifolia plant of West Africa, and is harvested specifically for it's 5-htp content. The amino acid 5-htp is commercially available as an oral supplement, and is also sold by wholesalers as a bulk powder.

### GABA

It is an object of this invention, the relevant inclusion of the amino acid GABA (gamma-aminobutyric acid) to the composition. Co-administration of GABA is an adjunct to 5-HTP, contributing both, synergistic and independent effects. GABA, like serotonin, is one of the neurotransmitters absorbed intestinally by enteric neurons (Tsai L H., 2005). As such, sublingual preparation prevents neuronal activation or metabolism in the periphery by the ENS, increasing the systemic availability to the CNS. The human pineal gland, a circumventricular organ outside the blood-brain-barrier, has several GABA-A receptor complexes that are capable of neurotransmitter uptake by glial cells and pinealocytes. Both, pre- and postsynaptic activity of GABA in the pineal does not differ from that found for GABA interneurons in local circuits of the brain (Rosenstein et al., 1990).

As to it's synergistic effects with 5-HTP, GABA further increases serotonin by decreasing it's metabolism to melatonin via pineal gland GABA-A receptor signaling (Rosenstein et al., 1989). As to it's independent effects, GABA is a neuromodulator of the intracortical processes underlying migraine aura. GABA, like serotonin is a principal inhibitory neurotransmitter of the central nervous system, with both analgesic and Anxiolytic effects. Activating GABA-ergic networks acts as the primary inhibitory mechanism in the visual cortex (Palmer et al., 2000). Additionally, the facilitation of GABA transmission at the GABA-A receptor results in potent inhibition of trigeminovascular nociceptive transmission (Storer et al., 2004).

It is therefore not uncommon for anti-epileptic GABA-ergics to be prescribed as migraine prophylactics (U.S. Pat. No. 5,767,117), (Zaremba et al., 2006). It is also postulated, that a GABA deficiency may be an underlying mechanism of depression in chronic migraine (Vieira et al., 2006).

### Vitamin Cofactors

It is an object of this invention, that the enzymatic synthesis of serotonin requires vitamin cofactors. And if the cofactors are not present within the preparation, neurotransmitter synthesis is limited by the CNS tissue levels of these vitamins. The provision then of these cofactors added to the preparation, prevents an ineffectual or incomplete neurotransmitter synthesis due to coenzyme insufficiency (U.S. Pat. Appl. No. 20060178423). It also overcomes the inter-individual differences of vitamin deficiency, rendering consistent and reproducible results. Water soluble vitamins are passively absorbed by osmosis, and the vitamin molecules are easily transported across the mucosal membranes. There are currently many oral preparations of 5-HTP on the market now, most being sold without vitamin cofactors. The inclusion of these vitamins within the invention are as follow:

Vitamin B3 (niacinamide) acts as a negative feedback regulator on the kynurenine pathway to shunt tryptophan into the serotonin pathway, thus increasing plasma serotonin levels (Velling et al., 2003). Additionally, B3 causes cutaneous flushing that might abort the acute symptoms of migraine by vasodilating the intracranial vessels, thus preventing the subsequent vasoconstriction of the extracranial vessels (Velling et al., 2005). Niacinamide specifically has been demonstrated to increase GABA transmission and binding at the post-synaptic level (Fomenko et al., 1993).

Vitamin B6 (Pyridoxal-5′-phosphate) is a coenzyme in the metabolism of both 5-HTP to serotonin by enzyme L-aromatic amino acid decarboxylase, and glutamic acid to GABA by L-glutamic acid decarboxylase (GAD). It also increases the peripheral uptake of tryptophan to the brain, while decreasing it's liver clearance (Bender et al., 1984).

Vitamin C enhances Tetrahydrobiopterin bioavalability, a coenzyme of tryptophan-5-hydroxylase, and is required for the conversion of tryptophan to 5-hydroxytryptophan in serotonin production (Cooper J R., 1961). Another very important function of vitamin C, that necessitates it's inclusion within the embodiment, is as a carrier acid. As 5-HTP and GABA alone are not quickly absorbed transbuccally, ascorbic acid lowers salivary pH, and increases the oral permeability of the 5-HTP preparation. The sour taste (acidity) of vitamin C increases salivary gland output, which enhances vasodilation, sublingual gland blood flow, and resultantly, circulatory uptake.

### Inactive Ingredients

Commercial flavor additives, obtainable in a concentrated powder form, may be added or combined as desired, to produce a particular flavor mix which is compatible with the other components, such that a good tasting confection is produced. Sweeteners such as aspartame, compressible confectioner's sugar, fructose, and Maltodextrin may also be acceptable for use within the scope of the present invention. Since the flavorings described are generally a white powder, as are the other active components, additional artificial colorings may also be added to the composition in order to produce a desirable color or commercially distinguishable product.

In another embodiment within the scope of the present invention, the active and inactive constituents may be combined to form a compressed powder drug delivery system. In which case, the addition of lactose, starch, or suitable carbohydrate, may be added into the matrix as needed to provide bulk and filling to sustain size requirements of a complete dose. Once the desired constituents are thoroughly mixed, they are compressed into a solid mass under high pressure. Typically, compressive forces in the range from approximately 2,000 Newtons to approximately 5,000 Newtons are preferred. As a result, the compressed powdered matrix is held together by physical means rather than by chemical means. The extent of the compressive forces can be modified to vary the rate that the medicament will dissolve in a patient's mouth. The greater the compressive forces that form the mixture, the slower the dissolution of the compressed powder matrix in the mouth (U.S. Pat. No. 4,863,737).

In yet another embodiment within the scope of the present invention, the active and inactive constituents may be combined with hydrogels or gelatins to form a dissolvable drug delivery system.

What is claimed is:

1. A method for treating a migraine in a human which comprises: administering to said human an amino acid and vitamin composition, said amino acid being a central nervous system neurotransmitter or neurotransmitter precursor, said vitamins being cofactors in neurotransmitter biosynthesis, composition being administered in an amount effective to provide an amelioration of acute migraine pain.

2. The method of claim 1 wherein said acute pain of a migraine of said human is from migraine with aura.

3. The composition of claim 1 further comprising at least one amino acid selected from the group consisting of 5-Hydroxy L-Tryptophan (5-HTP) and Gamma-Aminobutyric Acid (GABA).

4. The composition of claim 3 wherein said amino acid is 5-HTP in the amount of 10 mg to 300 mg.

5. The composition of claim 3 wherein said amino acid is GABA in the amount of 10 mg to 300 mg.

6. The composition of claim 1 further comprising at least one vitamin cofactor selected from the group consisting of vitamin B3, vitamin B6, and vitamin C.

7. The composition of claim 6 wherein said vitamin is vitamin B3 in the amount of 5 mg to 200 mg.

8. The composition of claim 6 wherein said vitamin is vitamin B6 in the amount of 2 mg to 50 mg.

9. The composition of claim 6 wherein said vitamin is vitamin C in the amount of 20 mg to 1000 mg.

10. The method of claim 1 wherein said composition being administered is by sublingual delivery.

11. The method of claim 10 wherein said composition being administered by sublingual delivery is in the group of powder, pill, liquid, and gelatin paste.

12. The method of claim 1 wherein said human is a male adult or child.

13. The method of claim 1 wherein said human is a female adult or child.
